(19) United States
(12) Patent Application Publication
(10) Pub. No.: US 2007/0281350 A1
(43) Pub. Date: Dec. 6, 2007

(54) Bacteria trapping apparatus

(76) Inventors: Hisao Inami, Matsudo (JP); Masahiro Kurihara, Yokohama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/236,788

(22) Filed: Sep. 28, 2005

(30) Foreign Application Priority Data
Jun. 13, 2005 (JP) 2005-171831

(51) Int. Cl. C12M 1/34

(52) U.S. Cl. 435/288.5; 435/297.2; 435/309.1

(57) ABSTRACT

A bacteria trapping apparatus comprises a nozzle for introducing air, a trapping chamber disposed on an exit side of the nozzle, and a trapping material disposed inside the trapping chamber for trapping bacteria. The air introduced into the trapping chamber is discharged by a discharge unit towards the side opposite to the nozzle. The trapping material, a trapping container, and a holding member together constitute a trapping unit, which preferably comprises a bacteria trapping chip that can be mounted on analysis equipment.

The present application claims priority from Japanese application JP2005-171831 filed on Jun. 13, 2005, the content of which is hereby incorporated by reference into this application.

### BACKGROUND OF THE INVENTION

The present invention relates to an apparatus for trapping airborne bacteria, particularly to a portable apparatus for trapping airborne bacteria.

Conventional methods for trapping bacteria suspended in the atmosphere include the bubbling method, filtering method, and collision method, for example. In the bubbling method, bacteria are blown into trapping water so that the bacteria can be suspended in the water. This method employs no filter and therefore there is no risk of clogging. In addition, because the bacteria are fed into water, there is no need for the step of separating bacteria that became attached to the trapping unit. However, this method requires the step of evaporating the water for condensing purposes. An example of the bubbling method is disclosed in JP Patent Publication (Kokai) No. 7-67694 A (1995). The filtering method employs a membrane, such as a high efficiency particulate air (HEPA) filter, for trapping bacteria. By adjusting the opening of the membrane, trapped bacteria can be classified, and the separation-limit grain size is advantageously small at 0.3 μm. The filtering method, however, is disadvantageous in that the membrane becomes clogged over time, and that it is difficult to separate the trapped bacteria from the membrane. An example of the filtering method is disclosed in JP Patent Publication (Kokai) No. 4-218393 A (1992).

The collision method involves a high-speed ejection of intake air through a nozzle portion so as to trap bacteria at a collision plate disposed downstream of the nozzle. The bacteria in the air are given a force of inertia proportional to the square of the grain size of each bacterium, before they become attached to the collision plate. In this method, there is no problem of clogging as in the filtering method and is advantageous in that bacteria can be collected in a condensed manner. Examples of the collision method is disclosed in JP Patent Publication (Kokai) No. 5-322739 A (1993) and JP Patent Publication (Kokai) No. 2000-125843 A.

In the collision method disclosed in JP Patent Publication (Kokai) No. 5-322739 A, a metal plate is disposed immediately below the nozzle as a trapping unit. Such a metal plate, which is smooth, has the problem of the bacteria that have become once attached tending to be separated due to the flow of air, thereby reducing the trapping rate.

In the collision method according to JP Patent Publication (Kokai) No. 2000-125843 A, a culture medium is disposed immediately below a plurality of minute nozzles. However, the multiple nozzles prevent the bacteria from being condensed and instead cause them to be trapped in a dispersed manner. In a method for detecting trapped bacteria quickly and with high sensitivity, gene of a particular bacterium is amplified and detected. However, the bacterial gene cannot be detected efficiently unless the bacterium can be trapped at one location in a condensed manner. If the bacterial gene detection sensitivity were to be improved, a condensation operation would have to be performed, which would result in complicating the analysis procedure.

### BRIEF SUMMARY OF THE INVENTION

It is an object of the invention to provide a bacteria trapping apparatus capable of trapping airborne bacteria efficiently.

The bacteria trapping apparatus of the invention comprises a cap portion, a nozzle portion, a primary filter, a trapping unit, a support plate, a secondary filter, a fan motor, and a casing. The trapping unit includes a trapping material, a trapping container, and a holding member. The trapping unit may be comprised of a bacteria trapping chip adapted to be mounted on analysis equipment.

In accordance with the invention, bacteria suspended in the atmosphere can be trapped efficiently and then subjected to the detection of bacterial gene in a condensed state. The invention also provides a bacterial spore processing chip or apparatus that allows extraction of gene from a bacteria spore to be performed on a chip safely and in a short period of time.

### BRIEF DESCRIPTION OF THE SEVERAL VIEWS OF THE DRAWINGS

FIG. 1 shows an exploded perspective view of an embodiment of a bacteria trapping apparatus according to the invention.

FIG. 2 shows a lateral cross section of a major portion of FIG. 1.

FIG. 3 shows a partial detailed cross section of FIG. 2.

FIG. 4 shows a graph indicating trapping efficiency.

FIG. 5 shows a graph illustrating the result of an experiment.

FIG. 6 shows a graph illustrating the result of another experiment.

FIG. 7 shows an example of a trapping material used in the bacteria trapping apparatus shown in FIG. 1.

FIG. 8 shows trapping methods.

FIG. 9 shows a flowchart of a trapping method according to the invention.

FIG. 10 shows a top plan view of a trapping chip used in the trapping apparatus shown in FIG. 1.

FIG. 11 shows a longitudinal cross section of the trapping chip.

### DETAILED DESCRIPTION OF THE INVENTION

The invention will be hereafter described by way of example with reference to embodiments thereof.

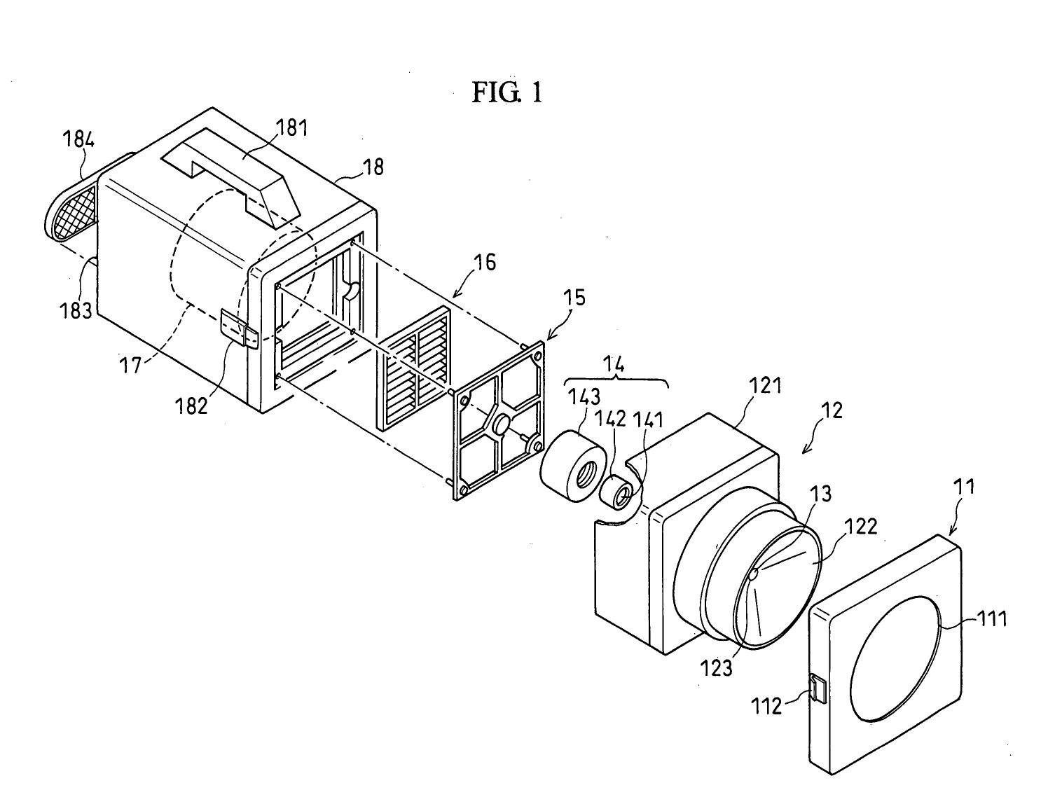

With reference to FIG. 1, an example of a bacteria trapping apparatus of the invention is described. FIG. 1 shows an exploded view of the bacteria trapping apparatus of the present example, which comprises a cap portion 11, a nozzle portion 12, a primary filter 13, a trapping unit 14, a support plate 15, a secondary filter 16, a fan motor 17, and a casing 18. The cap portion 11 is comprised of a square or rectangular member having a circular opening 111 and a fixing means 112 on either side thereof.

The nozzle portion 12 includes a square or rectangular box portion 121 and a columnar nozzle 122 mounted on the box portion. The primary filter 13 is disposed in a nozzle opening 123. The trapping unit 14 consists of a trapping material 141 for trapping bacteria, a trapping container 142 for housing the trapping material, and a holding member 143 for holding the trapping container. The holding member 143 is detachably supported by the support plate 15. The details of the trapping material 141 will be described later.

When the bacteria trapping apparatus is assembled, the secondary filter 16, the support plate 15 to which the trapping unit 14 is attached, the nozzle portion 12 to which the primary filter 13 is attached, and the cap portion 11 are sequentially inserted into a recessed portion of the casing 18 at the front thereof. The fixing means 112 of the cap portion 11 and a fixing means 182 of the casing are then engaged with one another. A battery is installed inside the casing. A handle portion 181 is provided at the top of the casing. At the rear of the casing, an exhaust outlet 183 is provided, in which a cap 184 with a mesh is fitted.

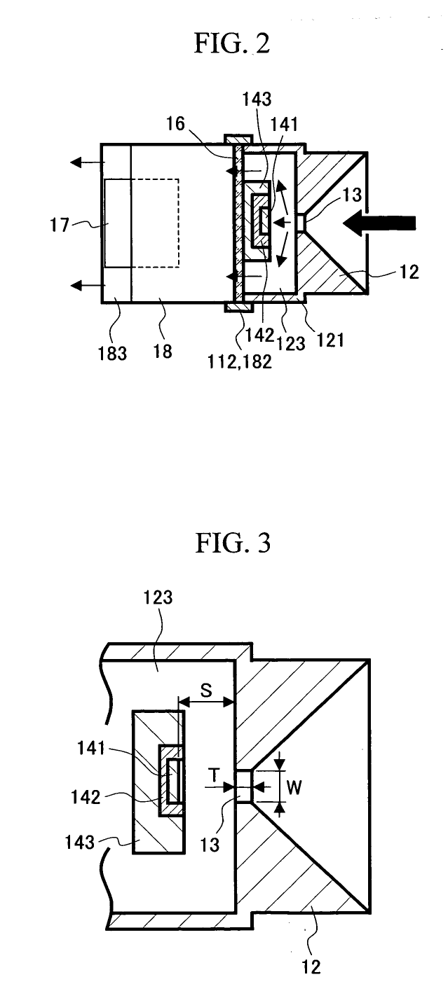

With reference to FIGS. 2 and 3, the operation of the bacteria trapping apparatus of the example is described. FIG. 2 schematically shows a cross section of a major portion of the bacteria trapping apparatus of the example. FIG. 3 schematically shows a cross section of a front-edge of the major portion of the bacteria trapping apparatus. As shown, a trapping chamber 123 is formed inside the apparatus by the box portion 121 of the nozzle portion 12. Within the trapping chamber 123, the trapping material 141, trapping container 142, and holding member 143 are disposed.

When the fan motor 17 is driven, air is sucked through the nozzle portion 12. The intake air is accelerated by the nozzle and passes through the primary filter 13, and it is eventually introduced into the trapping chamber 123. Coarse particles in the air are removed by the primary filter 13. Fine particles in the air introduced into the trapping chamber 123 collide with the trapping material 141 with inertia and become attached thereto. The air introduced into the trapping chamber 123 then passes through the secondary filter 16, as shown by the arrow, and is then discharged to the outside via the exhaust outlet 18 at the rear of the casing. Fine particles that have not been trapped by the trapping material 141 are removed by the secondary filter 16.

The trapping container 142 in the trapping unit 14 is discarded after one use and replaced with a new one. The trapping container 142 is preferably made of a resin material such as AS resin (acrylonitrile-ethylenepropylene-styrene-copolymer), ABS (acrylonitrile-butadiene-styrene-copolymer), PP (polypropylene), LDPE (low-density polyethylene), HDPE (high-density polyethylene), PET (polyethylene terephthalate), PC (polycarbonate), PS (polystyrene), or PMMA (polymethylmethacrylate), for example. The trapping container 142 needs to be inexpensive and resistant to the processing temperature (60° C.) in a subsequent enzymatic treatment of bacteria. For these reasons, PP (polypropylene) or PS (polystyrene) is most suitable.

The primary filter 13 and the secondary filter 16 are washed or replaced with new ones as needed. Portions that come into contact with the flow of atmosphere, such as the inside surfaces of the nozzle portion 12 and trapping chamber 123, as well as the trapping container 142 and the holding member 143, are preferably made of or coated with polytetrafluoroethylene resin for reasons of charging as will be described later. Obviously, the recessed portion of the casing, the trapping container and the holding member in the trapping unit, the support plate, the trapping unit, the nozzle portion and the cap portion are preferably made of or coated with polytetrafluoroethylene resin for reasons of durability as well.

The tendency of materials to be positively charged by static electricity is sometimes expressed by an indicator referred to as a triboelectric series. A triboelectric series lists, for example, air, glass, nylon, silk, aluminum, paper, iron, rubber, polyethylene, silicon, and polytetrafluoroethylene resin in order of decreasing tendency to charge positively. Thus, it can be seen that polytetrafluoroethylene resin tends to be negatively charged. Bacteria, meanwhile, are also negatively charged. Therefore, polytetrafluoroethylene resin and bacteria are likely to be charged with the same polarity, and it is unlikely that bacteria become attached to polytetrafluoroethylene resin.

Polytetrafluoroethylene resin is also resistant to high temperatures and pressures, and it can, for example, be sterilized at high temperature of 120° C. Therefore, by sterilizing the nozzle portion 12, the trapping container 142 of the trapping unit 14, and the holding member 143 in advance, the bacteria that have become attached to them can be prevented from being carried over.

The primary filter 13 is provided for trapping coarse particle in the atmosphere, as mentioned above. The mesh opening of the filter is preferably 100 to 200 μm. In a period when the amount of airborne pollens increases, the mesh opening is preferably 10 to 100 μm. In this way, pollens, whose particle size is 10 μm or greater, and bacteria, whose particle size is less than 10 μm, can be simply classified. The primary filter 13 is preferably made of stainless steel or polytetrafluoroethylene resin, for ease of washing and high-temperature sterilization.

The secondary filter 16 is provided for preventing the fine particles of bacteria or the like that were not trapped by the trapping unit 14 from being emitted into the atmosphere via the exhaust outlet 183, as mentioned above. The secondary filter 16 is preferably comprised of a HEPA (high efficiency particulate air) filter, which is capable of trapping 99.97% or more of fine particles of 0.3 μm or greater. More preferably, the secondary filter 16 is comprised of a ultra low penetration air (ULPA) filter, which is capable of trapping 99.999% or more of fine particles of 0.1 to 0.2 μm. When a ULPA is used, the level of cleanness of the air released into the atmosphere via the exhaust outlet 183 can be further increased.

The trapping efficiency of the bacteria trapping apparatus of the present example is described with reference to FIG. 3. As shown, when the internal diameter of the nozzle opening is W, the size of the nozzle opening in the axial direction is T, and the distance between the nozzle and the trapping material 141 is S, a trapping efficiency η is arranged using a Stalks number Sk^{0.5} as a parameter, which is a dimensionless number and is expressed by the following equation:
Sk^{0.5}=(ρd²u/μW)^{0.5} (1)
where ρ is the density of particle, d is particle diameter, u is wind velocity, μ is the viscosity of air, and W is the internal diameter of the nozzle. Since bacteria are more than 98% water, the particle density is assumed to be 1 g/cm³, and the intake flow rate is assumed to be 100 L/min, and the internal diameter W of the nozzle is assumed to be 10 mm. Substituting ρ=103 kg/m³, d=1.5×10⁻⁶ m, u=220 m/s, μ=1.5×10⁻⁶ Pa·s, and L=3×10⁻³ m into Equation (1), we get Sk^{0.5}=2.9.

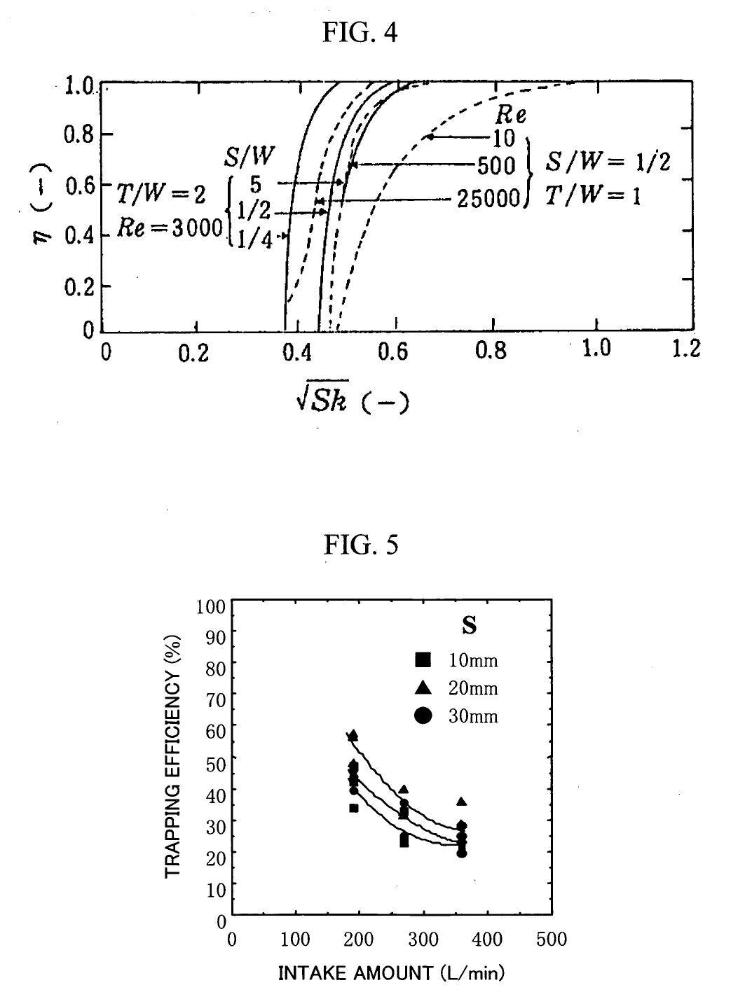

FIG. 4 shows a quotation from a prior-art publication regarding the trapping efficiency η (dimensionless number) and the Stokes number Sk^{0.5} (“Aerosol Technology: Limit particle size of an impactor and a trapping efficiency,” Inoue-shoin Publishing 1985-4). As shown, the point where Sk^{0.5}=2.9 is located well towards the right of the trapping efficiency curve, indicating that theoretically 100% of fine particles are trapped.

The internal diameter W of the nozzle opening is greatly related to the trapping efficiency. When the internal diameter W of the nozzle opening is smaller than 10 mm, bacteria can be trapped in a condensed manner. However, the air flow rate through the nozzle portion 12 increases, resulting in an increase in pressure loss, which increases in proportion to the square of the air flow rate. As a result, the load to the fan motor 17 increases, and the voltage of the driving battery drops. For example, when the internal diameter W of the nozzle opening is 3 mm or less, the work volume that can be handled by the battery (Li-MH) that can be mounted on a portable bacteria trapping apparatus would be exceeded. Therefore, the internal diameter W of the nozzle opening should be 4 to 15 mm. More preferably, it is 8 to 12 mm. In this way, bacteria can be trapped in a condensed manner immediately below the nozzle while a high trapping efficiency is achieved.

With reference to FIG. 5, the trapping efficiency of the bacteria trapping apparatus of the invention is described. One parameter that affects the trapping efficiency of the bacteria trapping apparatus is the interval S between the nozzle opening and the trapping material 141, as well as the internal diameter W of the nozzle opening. FIG. 5 shows the result of measuring the trapping efficiency while the interval S between the nozzle opening and the trapping material 141 was varied between 10 mm and 30 mm, where the vertical axis shows the trapping efficiency and the horizontal axis shows the intake amount. As shown, a maximum trapping efficiency is achieved when the interval S is 20 mm. When the interval S is 10 mm, the trapping efficiency drops due to the increase in the frequency of the sample that has once become trapped by the trapping material 141 being separated by air flow.

As described above, the internal diameter W of the nozzle opening as a parameter affecting the trapping efficiency is preferably 8 to 12 mm, and the interval S between the nozzle opening and the trapping material 141 is preferably 20 mm. Thus, their ratio S/W is preferably 2 to 3, and more preferably 2.

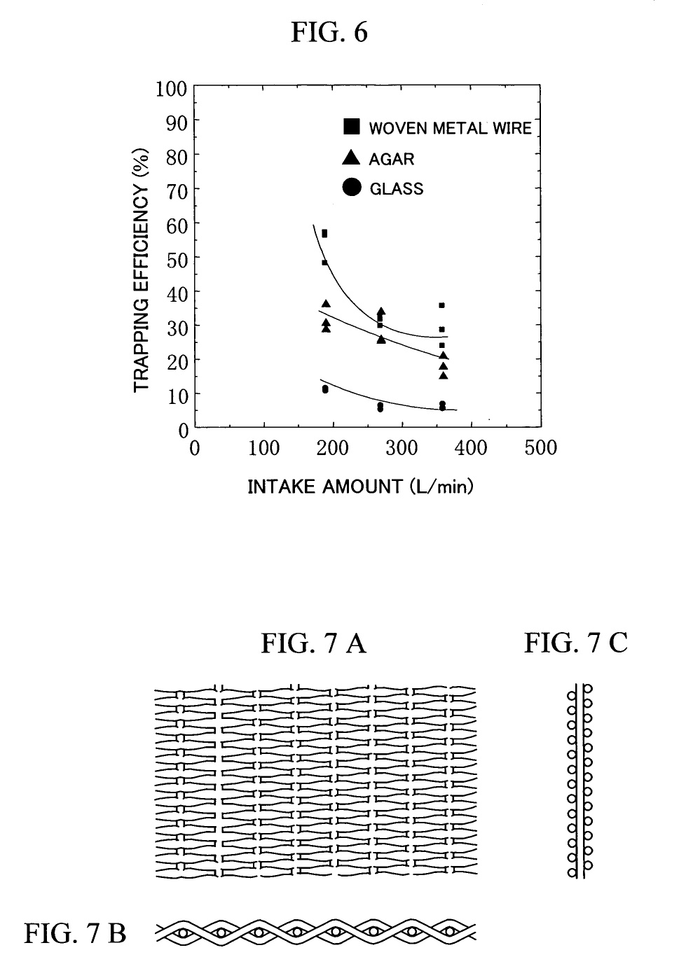

With reference to FIG. 6, the material of the trapping material 141 is described. FIG. 6 shows the result of measuring the trapping efficiency in the case of Bacillus subtilis when three kinds of material, namely woven metal wire, glass, and an agar medium were used as the trapping material 141. It can be seen that the woven metal wire provides the highest trapping efficiency.

When the material has a smooth surface, such as in the case of a metal plate, the bacteria that have once become attached tend to be separated by airflow, resulting in a lower trapping efficiency. Therefore, the trapping material 141 is preferably comprised of a material with large surface irregularities. The surface of woven metal wire has large irregularities and is therefore suitable for the trapping of fine particles. However, if the height of the surface irregularities exceeds 0.5 mm, it would be difficult to remove the fine particles that have become lodged in the mesh irregularities. Thus, the height of the surface irregularities on the woven metal wire is preferably 0.5 mm or smaller.

Among woven metal wires, those woven with larger mesh (the number of mesh openings per inch) are more preferable. For example, Dutch weave is better than plain weave because Dutch weave has very small openings in the mesh and a large surface with which bacteria can be trapped.

FIG. 7(a) shows a plan view of the surface configuration of Dutch weave. FIGS. 7(b) and (c) show cross sections. When the mesh is identical, a woven metal wire with Dutch weave has approximately twice as large surface area as a plain-woven metal wire, resulting in a greater bacteria trapping efficiency.

Although glass has a flat surface, it tends to charge positively. Because bacteria tend to charge negatively, bacteria can be trapped using the charge property of glass.

Of the aforementioned materials, the agar medium provided the second highest trapping efficiency, following the woven metal wire. One characteristic of the agar medium was that it has an adhesive property produced by the free water on the gel surface (the water in the gel mesh openings).

The concentration of agar in the agar medium is preferably 2 to 5%, and more preferably 3 to 4%. If the agar concentration is less than 2%, the water content would be excessive and the strength of the agar as a trapping material, against which high-speed air would continuously collide, would be lacking. On the other hand, if the agar concentration is greater than 6%, the water (free water) content on the agar surface would be too little, resulting in a significant drop in its adhesive property.

As a means of enhancing the trapping efficiency of the agar medium, a woven metal wire structure could be formed on the surface of the agar medium. For example, when the configuration of a Dutch-woven metal wire with the mesh opening of 20 μm was transferred to the surface of an agar medium of a 3% concentration and Bacillus subtilis was trapped using the agar medium, the trapping efficiency value was approximately twice as much as when the surface of the agar medium was flat. Thus, it can be said that the agar medium to which the configuration of a Dutch-woven metal wire has been transferred is most suitable as the trapping material 141 with a high trapping efficiency.

Conventionally, when bacteria trapped from the atmosphere are detected, the number of colonies grown by culturing the bacteria is counted. However, this method requires that the bacteria be cultured for more than one night before colonies that can be visually detected are obtained. In another method for highly sensitively and quickly detecting the trapped bacteria, gene of the bacteria is amplified and detected.

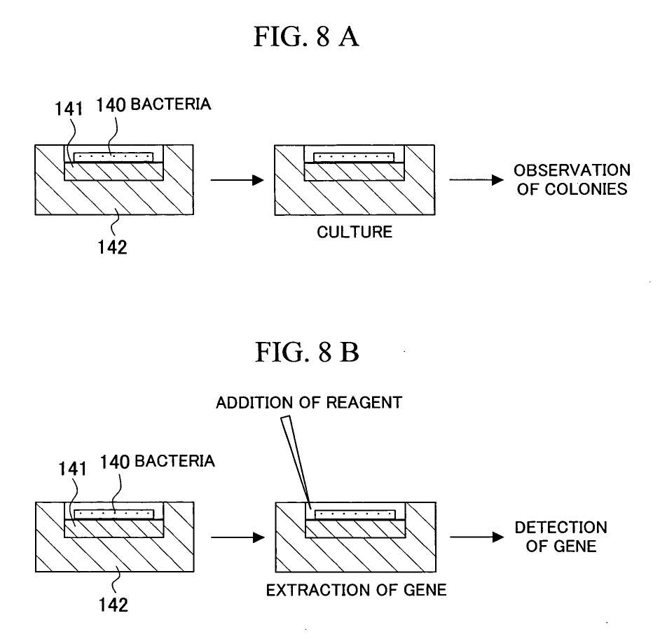

As shown in FIG. 8, when an agar medium is used as the trapping material 141, not only the trapped bacteria can be directly detected in the trapping material 141, but also gene of the bacteria can be advantageously detected. FIG. 8A shows a method of culturing a bacterium 140 as trapped in the agar medium as the trapping material 141 and identifying the bacterium based on the observation of its colonies. By adding peptone, alanine, or the like in the agar medium for bacterial cultivation purposes in advance, the efficiency of bacterial cultivation can be enhanced. FIG. 8B shows a method of detecting a bacterial gene using a part of the bacterium 140 trapped in the agar medium as the trapping material 141.

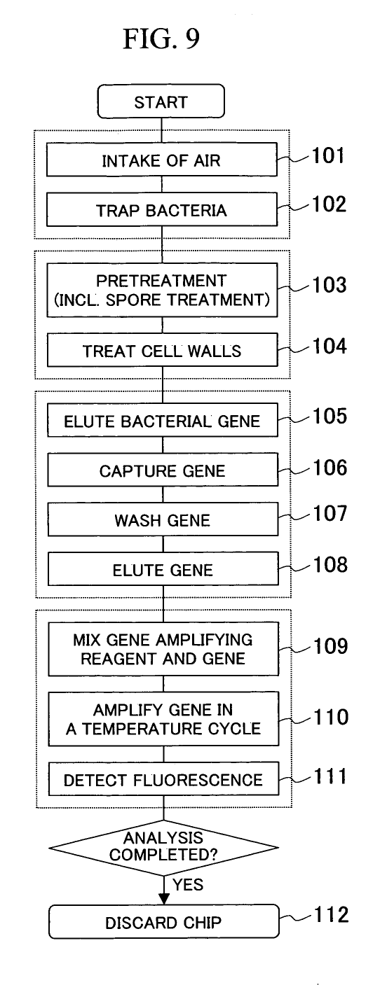

With reference to FIG. 9, a method of detecting the bacterial gene is described. At step 101, air is sucked through the nozzle portion of the bacteria trapping apparatus. At step 102, bacteria are caused to become attached to the trapping material 141 of the bacteria trapping apparatus. The volume of intake air is approximately 1000 L, for example.

At step 103, the trapping container 142 to which the trapping material 141 is attached is removed from the holding member 143, and a gene detection pretreatment is performed. The “pretreatment,” which herein mainly refers to the extraction of gene from the bacteria, would also include the processing of spores in cases of bacteria of the genus Bacillus, such as anthrax. This is due to the fact that the spore is a very hard shell-like material and has a very strong resistance to heat, chemical substance, or ultraviolet rays. For this reason, 100 μL of a germination promoter is added to the trapping material 141. Examples of the germination promoter include a bouillon containing alanine, adenosine, and glucose. Particularly, a bouillon containing 1 mM to 10 mM of L-alanine is most suitable. After 10 minutes or more, the bacterial spore would start germination. After 30 minutes or more, 80% of the entirety would germinate. Temperature for germination of the bacterial spore is preferably 35 to 40° C. and more preferably 35 to 37° C. When the bacterial spore geminates, the bacteria destroy their own spores, such that the cell walls of the bacteria would be exposed upon germination.

Next, at step 104, the cell walls of the bacteria are destroyed by an enzymatic process. Specifically, a protein denaturing enzyme for destroying the cells walls is added to the trapping material 141 in a sequential manner, and the temperature is maintained at an optimum temperature. This enzymatic process is preferably performed more than 10 minutes, more preferably 30 minutes. Preferable examples of the protein denaturing enzyme include 100 μL of lysozyme (optimum temperature: 37° C.) and 10 μL of protease K (optimum temperature: 55 to 60° C.). When 100 μL of chaotropic ion, such as guanidine thiocyanate, guanidine hydrogen chloride, sodium iodide, or potassium bromide is added to protease K, the activity of protease K can be improved. After these enzymatic processes, the cell membranes of the bacteria in the trapping material 141 are exposed.

At step 105, the bacterial gene is eluted. Specifically, by adding a cell membrane dissolving solution to the trapping material 141 having a suspension containing the bacteria with their cell membrane destroyed, the cell membrane of the bacteria is destroyed, and the gene of the bacteria is released outside the cells. Examples of the cell membrane dissolving solution include a solution containing a chaotropic ion, such as guanidine thiocyanate, guanidine hydrogen chloride, sodium iodide, or potassium bromide.

At step 106, the gene is captured using a gene-binding carrier. At step 107, remaining proteins and chaotropic ion are removed using a washing solution. At step 108, the gene captured on the gene-binding carrier is eluted using an eluant. Examples of the eluant include sterile distilled water, and a buffer solution such as TRIS-EDTA and TRIS-acetate. Using the thus extracted sample, gene is detected on a gene detection apparatus in accordance with a procedure as described below, for example.

At step 109, 5 μL of a gene amplifying reagent is mixed with the gene. The gene amplifying reagent is comprised of four kinds of 2.5 mM concentration of dNTP (dATP, dCTP, dGTP, dTTP), a buffer (100 mM concentration of TRIS hydrochloric acid, 500 mM concentration of KCl, and 15 mM concentration of MgCl 2), two kinds of primers, a DNA synthetase (TaqDNA polymerase, Tth DNA polymerase, Vent DNA polymerase, or ThermoSequenase), fluorescent dye (ethidium bromide or SYBR GREEN (from Molecular Probe Inc.).

At step 110, the gene is amplified in a temperature cycle such that the temperature is changed between two kinds of set values in a reciprocating manner as shown below.

The following is an example of the temperature cycle:

“90 to 95° C. for 10 to 30 sec [symbol] 65 to 70° C. for 10 to 30 sec”: 30 to 45 times

The following is a more preferable example:

“94° C. for 30 sec [symbol] 68° C. for 30 sec”: 45 times

At step 111, the gene is irradiated with an excitation light while the temperature cycle is repeated. When the gene has intercalated fluorescent dye inside its two chains, the fluorescent dye is excited and it emits fluorescence. Namely, if the sample has a target gene, the amount of fluorescence increases as the gene is amplified. Therefore, by monitoring the amount of fluorescence from the gene during the temperature cycle, the presence or absence of the target gene can be detected on real time.

The above-described steps (105 to 111) for extracting gene, amplifying it, and detecting it may be performed on analysis equipment using a bacteria analysis chip.

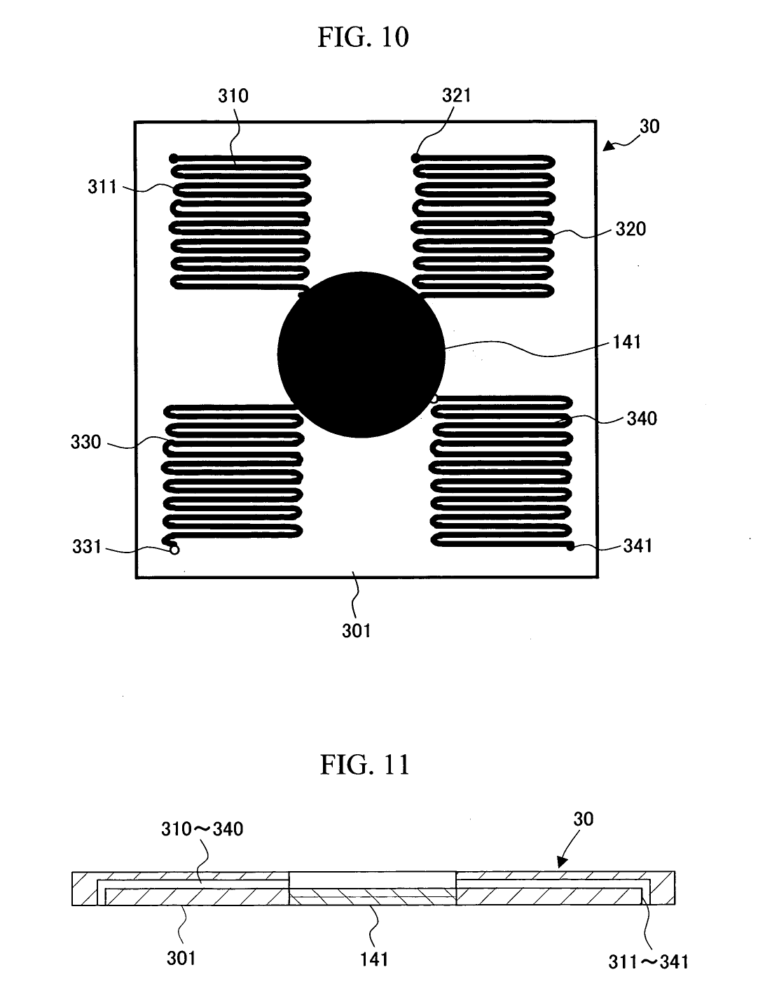

With reference to FIGS. 10 and 11, another example of the trapping unit of the bacteria trapping apparatus of the invention is described. The trapping unit of the present example is comprised of a bacteria analysis chip for use on analysis equipment. After bacteria have been trapped by the bacteria trapping apparatus, the trapping unit can be detached and set on the analysis equipment. In the following description, the trapping unit will be referred to as a trapping chip.

FIG. 10 shows a plan view of a trapping chip 30, and FIG. 11 shows a cross section of the trapping chip 30. The trapping chip 30 includes a thin plate of a substrate 301, a trapping material 141, a germination promoter storage bath 310 for storing a germination promoter, an enzyme-A storage bath 320 for storing lysozyme, an enzyme-B storage bath 330 for storing protease K, and a chaotropic storage bath 340 for storing an chaotropic ion. These baths are comprised of channels. One end of these baths is connected to the trapping material 141, and the other end is connected to chip ports (311 to 341). The chip ports constitute contact points with external channels. The germination promoter storage bath 310, the enzyme-A storage bath 320, the enzyme-B storage bath 330, and the chaotropic storage bath 340 are in communication with the trapping material 141. Thus, the trapping material 141 is connected to external channels via the germination promoter storage bath 310, the enzyme-A storage bath 320, the enzyme-B storage bath 330, the chaotropic storage bath 340, and the chip ports.

The volume of the germination promoter storage bath 310 is preferably 20 to 100 μL, the volume of the enzyme-A storage bath 320 is preferably 20 to 100 μL, the volume of the enzyme-B storage bath 330 is preferably 5 to 10 μL, and the volume of the chaotropic storage bath 340 is preferably 2 to 100 μL. When an agar medium is used as the trapping material 141, the thickness of the agar medium is preferably 5 to 10 mm. If the thickness of the agar medium is less than 5 mm, the agar medium would be contracted by the wind pressure.

The trapping chip 30 is attached to the support plate 15 of the bacteria trapping apparatus and the airborne bacteria are trapped for a certain period of time. The trapping chip 30 is then removed from the support plate 15 and set on analysis equipment, and the germination promoter, lysozyme, protease K, and chaotropic ion, which are hermetically sealed inside the trapping chip 30 in advance, are delivered to the trapping material 141 at certain time intervals. In this way, the spores and cell walls of the bacteria that have become attached to the trapping material 141 of the trapping chip 30 can be processed. Namely, steps 101 to 104 described with reference to FIG. 8 can be automatically carried out on the trapping chip. Thus, in the present example, the reagent dispensing operation can be omitted. The activity of reagents can be maintained for a month as long as the unused trapping chip 30 is supplied to the user in a frozen state and the user preserves it in a frozen state at 0° C. If the trapping chip 30 is preserved in a frozen state at −20° C., the activity of reagents can be maintained for more than six months.

While preferred embodiments of the invention have been described using specific terms, such description is for illustrative purposes only, and it is to be understood that changes and variations may be made without departing from the spirit or scope of the following claims.

What is claimed is:

1. A bacteria trapping apparatus comprising:
a nozzle for introducing air;
a trapping chamber provided on an exit side of said nozzle;
a trapping material disposed in said trapping chamber for trapping bacteria; and
an exhaust unit for discharging the air introduced into said trapping chamber towards the side opposite to said nozzle.

2. The bacteria trapping apparatus according to claim 1, comprising a first filter disposed in an opening of said nozzle.

3. The bacteria trapping apparatus according to claim 2, comprising a secondary filter disposed in said exhaust unit of said trapping chamber.

4. The bacteria trapping apparatus according to claim 2, wherein the mesh opening of said first filter is larger than the mesh opening of said second filter.

5. The bacteria trapping apparatus, comprising a fan downstream of said exhaust unit of said trapping chamber, wherein said fan sucks the air inside said trapping chamber and delivers it to the outside via said exhaust unit.

6. The bacteria trapping apparatus according to claim 1, wherein the inner walls of said trapping chamber and/or said nozzle are made of or coated with polytetrafluoroethylene resin.

7. The bacteria trapping apparatus according to claim 1, wherein the internal diameter of the opening of said nozzle is 4 to 15 mm.

8. The bacteria trapping apparatus according to claim 1, wherein the interval between the opening of said nozzle and said trapping material is 10 to 30 mm.

9. The bacteria trapping apparatus according to claim 1, wherein the ratio of the internal diameter, W, of the opening of said nozzle to the interval, S, between the nozzle opening and said trapping material, or S/W, is 2 to 3.

10. The bacteria trapping apparatus according to claim 1, comprising a trapping container for storing said trapping material and a holding member for holding said trapping container.

11. The bacteria trapping apparatus according to claim 10, wherein said trapping container and said holding member are made of or coated with polytetrafluoroethylene resin.

12. The bacteria trapping apparatus according to claim 1, comprising a casing, wherein said nozzle and said trapping chamber are disposed in an opening portion of said casing, wherein an exhaust outlet is provided at the end of said casing opposite to said opening portion, and wherein said apparatus is constructed as a portable device.

13. A bacteria trapping apparatus comprising:
a nozzle for introducing air;
a first filter disposed in an opening of said nozzle;
a trapping chamber disposed on an exit side of said nozzle;
a trapping material disposed in said trapping chamber for trapping bacteria;
an exhaust portion for discharging the air introduced into said trapping chamber toward the side opposite to said nozzle; and
a secondary filter disposed in said exhaust portion.

14. The bacteria trapping apparatus according to claim 13, wherein said trapping material comprises a woven metal wire, wherein the height of the surface irregularities of said woven metal wire is 0.5 mm or less.

15. The bacteria trapping apparatus according to claim 13, wherein said trapping material is an agar medium with a concentration of 2 to 5%.

16. The bacteria trapping apparatus according to claim 13, wherein said trapping material is an agar medium having a surface with a woven metal wire pattern transferred thereon.

17. The bacteria trapping apparatus according to claim 12, wherein said trapping material is comprised of a trapping chip that can be mounted on analysis equipment, wherein said trapping chip comprises a trapping unit for trapping bacteria, a port connectable to a corresponding connector portion of said analysis equipment, and a storage bath in which a predetermined preparation is stored, wherein one end of said storage bath is connected to said trapping unit, and the other end is connected to said port.

18. The bacteria trapping apparatus according to claim 17, wherein said storage bath in said trapping chip includes:
a germination promoter storage bath for storing a germination promoter;
an enzyme storage bath for storing lysozyme;
another enzyme storage bath for storing protease K; and
a chaotropic storage bath for storing a chaotropic ion.

19. The bacteria trapping apparatus according to claim 18, wherein said germination promoter comprises a bouillon containing L-alanine.

20. A bacteria trapping chip comprising:
a plate-shaped substrate;
a trapping material disposed in a recessed portion at the center of said substrate for trapping bacteria;
a germination promoter storage bath for storing a germination promoter;
an enzyme-A storage bath for storing lysozyme;
an enzyme-B storage bath for storing protease K; and
a chaotropic storage bath for storing chaotropic ion,
wherein said germination promoter storage bath, said enzyme-A storage bath, said enzyme-B storage bath, and said chaotropic storage bath are comprised of channels formed in said substrate, one end of these baths being connected to said recessed portion where said trapping material is disposed and the other end of these baths being connected to a chip port for connection with external channels.
